(19) United States
(12) Patent Application Publication
(10) Pub. No.: US 2011/0098694 A1
(43) Pub. Date: Apr. 28, 2011

(54) METHODS AND INSTRUMENTS FOR TREATING CARDIAC TISSUE THROUGH A NATURAL ORIFICE

(75) Inventor: Gary L. Long, Cincinnati, OH (US)

(73) Assignee: Ethicon Endo-Surgery, Inc., Cincinnati, OH (US)

(21) Appl. No.: 12/607,388

(22) Filed: Oct. 28, 2009

(51) Int. Cl. A61B 18/02; A61B 18/14; A61B 18/18

(52) U.S. Cl. 606/21; 606/41; 606/33

(57) ABSTRACT

Ablation probes for ablating cardiac tissue through a patient's natural orifice and methods for ablating cardiac tissue through a patient's natural orifice. In some embodiments, an ablation probe, which may include electrodes, a radio frequency probe or a cryoprobe, is inserted through an incision made through the patient's esophagus. A balloon catheter may be inserted through the incision after it has been made to expand the area between the heart and the esophagus. The ablation probe is then brought into contact with the cardiac tissue and activated to irreversibly damage the tissue. The ablation probes may employ vacuum to retain the tissue in contact with the probe during ablation.

### BACKGROUND

The embodiments relate, in general, to surgical procedures and instruments for treating cardiac tissue and, more particularly, to instruments and methods involving the access to such cardiac tissue through a patient's natural orifice.

Minimally invasive procedures are desirable because such procedures can reduce pain and provide relatively quick recovery times as compared with conventional open medical procedures. Many minimally invasive procedures include those that are performed through insertion of an endoscope through a natural body orifice to a treatment region. Minimally invasive therapeutic procedures to treat or diagnose diseased tissue by introducing medical instruments translumenally to a tissue treatment region through a natural opening of the patient are known as Natural Orifice Translumenal Endoscopic Surgery (NOTES)™.

One area wherein the use of NOTES type instruments and procedures could be advantageously employed is in the treatment of cardiac tissue. For example, it has been found that atrial fibrillation is the most common irregular heart rhythm in the United States. Atrial fibrillation is an abnormal heart rhythm originating in the atria (top chambers of the heart). Such atrial fibrilation occurs when the heart impulses fail to travel in an orderly fashion through the heart and, instead, many impulses begin simultaneously and spread through the atria, which may lead to a rapid and disorganized heartbeat.

In the past, atrial fibrillation was believed to be a harmless annoyance. However, the medical profession has now recognized atrial fibrillation as a dangerous condition. Statistics show that atrial fibrillation may actually double the risk of death and may increase the risk of stroke five to seven times when compared with the person who does not suffer from atrial fibrillation. In addition, atrial fibrillation may cause congestive heart failure and uncomfortable symptoms related to a rapid heart rate.

One method that has been developed to treat atria fibrillation is commonly referred to as the “Cox-Maz” procedure. Such procedure involves the application of a series of precise incisions in the right and left atria to interrupt the conduction of abnormal impulses. Such incisions allow sinus implulses to travel to the atrioventricular node as they would under normal conditions. A transthoracic approach has been taken in the past to gain access to the posterior side of the heart.

Consequently a need exists for instruments that may be inserted through a patient's esophagus to treat cardiac tissues.

The foregoing discussion is intended only to illustrate some of the shortcomings present in the field at the time, and should not be taken as a disavowal of claim scope.

### SUMMARY

In one embodiment, an ablation probe assembly for treating cardiac tissue is provided. In various embodiments, the assembly includes a first elongated electrode probe that has a first polarity. The assembly may further include a second elongated electrode probe that has a second polarity that is opposite from the first polarity. The second elongated electrode probe may be coupled in spaced relation to the first elongated electrode probe by a first non-electrically conductive member to facilitate movement of the first and second elongated electrode probes relative to each other.

In another general embodiment there is provided a method for treating cardiac tissue. The method may include forming an incision through a patient's esophagus adjacent a posterior portion of the patient's heart and inserting an ablation probe through the incision in the esophagus. The method may further include activating the ablation probe to ablate a portion of heart tissue.

In yet other general embodiments, there is provided an ablation probe for ablating cardiac tissue. The ablation probe may comprise an elongated flexible housing that is sized to be inserted through a flexible catheter. The elongated flexible housing may be fabricated from non-electrically conductive material and have first and second separate vacuum passages formed therein. A first positive electrode may be disposed within the elongated flexible housing and a second negative electrode may also be disposed within the elongated flexible housing such that the first said vacuum passage separates the first and second electrodes. A third negative electrode may also be disposed within the elongated flexible housing such that the second vacuum passage separates the first and third electrodes.

In still another general aspect of the present invention there is provided an ablation probe for ablating cardiac tissue. In various embodiments, the ablation probe includes a first elongated electrode that has a first polarity and a second elongated electrode that has a second polarity that is opposite from the first polarity. A non-electrically conductive hinge member may be coupled to the first and second elongated electrodes such that the first and second elongated electrodes may be moved between a first position wherein the first and second electrodes are adjacent to each other in a non-coplanar relationship and a second position wherein the first and second electrodes are substantially coplanar with each other.

### BRIEF DESCRIPTION OF THE FIGURES

The novel features of the embodiments described herein are set forth with particularity in the appended claims. The embodiments, however, both as to organization and methods of operation may be better understood by reference to the following description, taken in conjunction with the accompanying drawings as follows.

FIG. 1 is a diagrammatical view illustrating deployment of an ablation device embodiment of the present invention through a patient's mouth;

FIG. 2 is a diagrammatical view of a patient's heart and esophagus illustrating deployment of a balloon catheter through an incision in the esophagus to expand the area between the heart and the esophagus;

FIG. 3 is another diagrammatical view of the heart illustrating deployment of an ablation probe of various embodiments of the present invention onto a portion of the heart;

FIG. 4 is a partial perspective view of another ablation probe assembly of the present invention;

FIG. 5 is a partial perspective view of another ablation probe assembly of the present invention;

FIG. 6 is a partial perspective view of another ablation probe assembly of the present invention in a folded state for insertion through a flexible catheter; and

FIG. 7 is another partial perspective view of the ablation probe assembly of FIG. 6 in a deployed open state on cardiac tissue.

### DETAILED DESCRIPTION

Certain embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting embodiments and that the scope of these embodiments is defined solely by the claims. The features illustrated or described in connection with one embodiment may be combined with the features of other embodiments. Such modifications and variations are intended to be included within the scope of the appended claims.

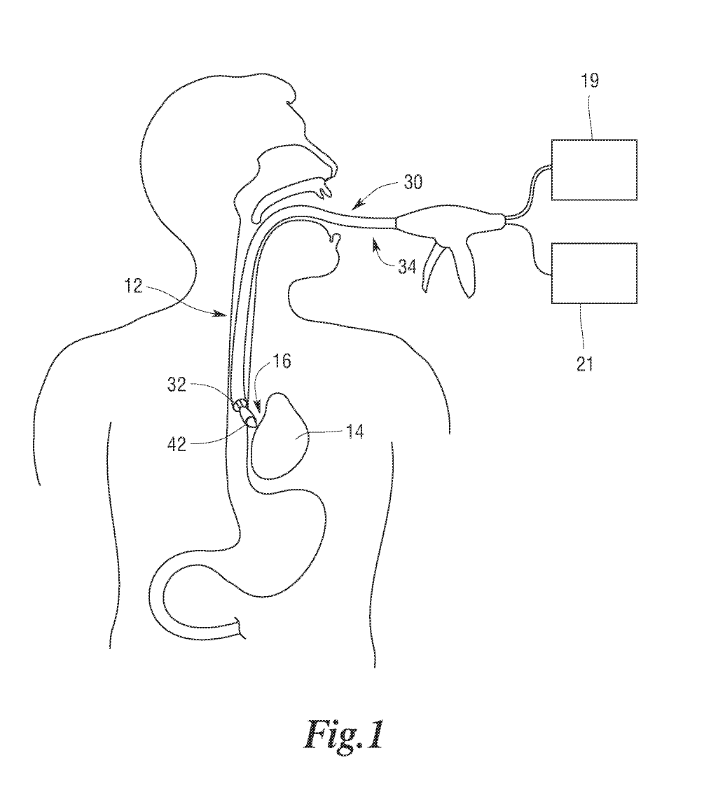

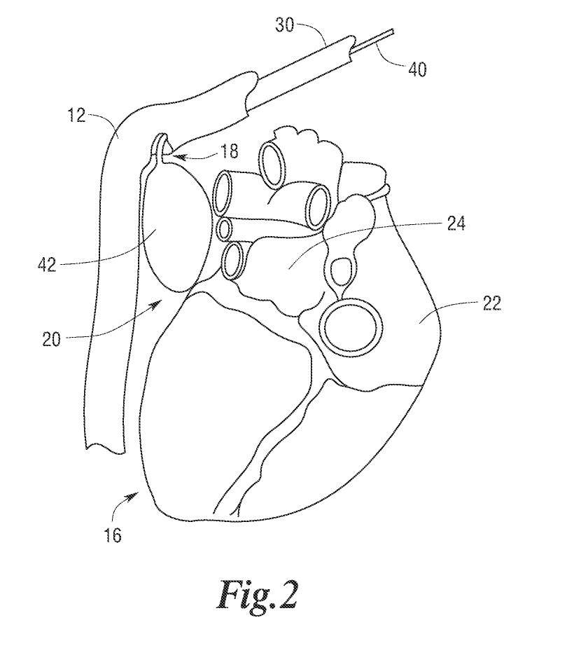

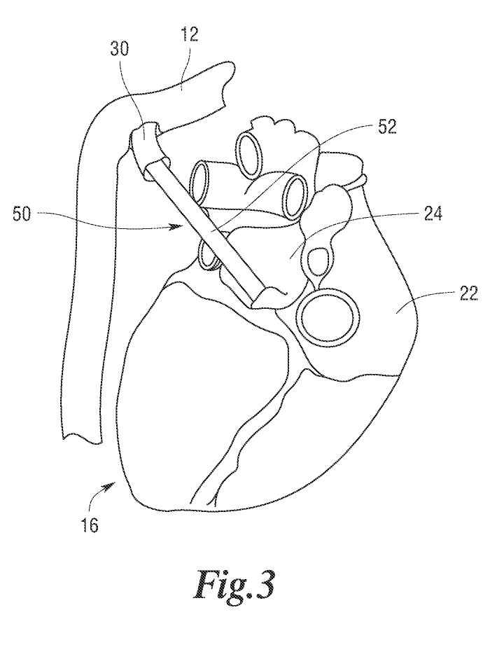

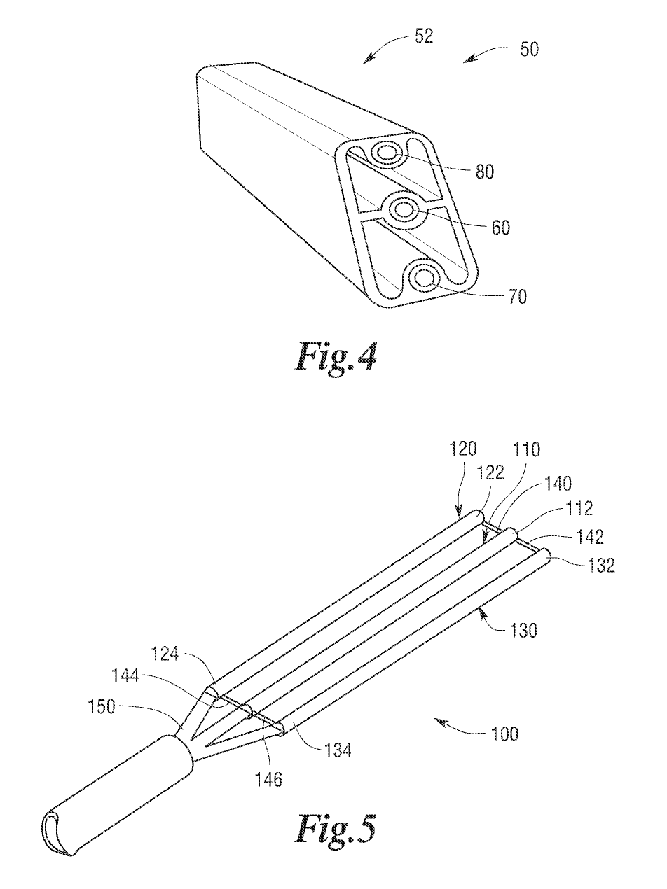

The various embodiments generally relate to “ablation probes” such as, for example, radio frequency “RF” probes, electrode configurations, cryogenic and other devices that may be operably deployed through a flexible catheter or working tube that is inserted through a patient's esophagus to treat cardiac tissue. FIG. 1 schematically illustrates a flexible catheter or working tube or endoscope 30 that has been inserted through a patient's mouth 10 and esophagus 12. The esophagus 12 is adjacent to the posterior side 16 of the heart 14. To gain access to the posterior side 16 of the heart 14, an incision 18 is made in the anterior side of the esophagus 12 by making an incision in the mucosa and submucosa of the esophagus, tunnel through space between the submucosa and muscular layer and make an excision through the muscular layer of the esophagus. This “offset” excision is commonly practiced. The distal end 32 of the flexible catheter 30 may then be advanced out through the incision 18 in the esophagus 12. The proximal end 34 of the flexible catheter 30 protrudes out of the patient's mouth 10 to enable the clinician to insert various instruments therethrough. For example, after the clinician has made the incision 18 through the esophagus 12, the clinician may then insert a catheter 40 with an inflatable balloon portion 42 therethrough such that the balloon portion 42 protrudes out through the incision 18. See FIG. 2. The balloon portion 42 is then inflated to expand the space 20 between the esophagus 12 and the heart 14 and expose the posterior portion of the heart 14 and the right and left atria 22, 24. See FIG. 2.

Once the clinician has gained access to the right and left atria 22, 24, the clinician may then insert an ablation probe to ablate the tissue on the right and left atria 22, 24 to interrupt the “bad conductive pathways” that result in atrial fibrillation. The ablation probe may comprise an RF device to thermally ablate the tissue, a cryoprobe to freeze the tissue or a DC pulse catheter to irreversibly damage the cells. For example, in various embodiments, the ablation device 50 may include an elongated flexible housing 52 that may be fabricated from a non-electrically conductive material such as, for example, Teflon®. The elongated housing 52 may be formed to non-movably to support three electrodes 60, 70 and 80. In one embodiment, for example, the two laterally-positioned, negative electrodes 70 and 80 are spaced from the centrally-disposed, positive electrode 60 by vacuum passages 56, 58 that are coupled to a source of vacuum 19 that may be located in the surgical suite. Thus, the first electrode 60 has a first polarity (positive) and the second and third electrodes 70, 80 each have a “second” polarity (negative) that is opposite to the first polarity. When employing the ablation probe assembly 50, the clinician may move the ablation probe assembly 50 into contact with the epicardium portion 23 of the left and right atria 22, 24 and then vacuum is applied to the passages 56 and 58 to hold the ablation probe assembly 50 in constant contact therewith. The electrodes 60, 70, 80 alternate in polarity, but do not penetrate the tissue physically. Pulses on the order of 3000 volts, 1.0 μseconds may be applied to the tissue 22, 24. Tissue damage which is relatively local around the end of the ablation probe assembly 50 may generally occur rapidly. Thus, the ablation probe assembly 50 may be advanced along the tissues 22, 24 to create linear regions as desired. For example, the depth of ablation achieved using the ablation probe assembly 50 may be 1.0 cm at the center.

FIG. 5 illustrates another ablation probe assembly 100 that may include three electrodes 110, 120, 130. Each electrode 110, 1120, 130 may be, for example, 1.0 cm long×0.5 cm wide×0.5 cm thick and be fabricated from stainless steel. A non-electrically conductive distal spacer member 140 extends between the distal end 112 of electrode 110 and the distal end 122 of the central electrode 120. Similarly, another non-electrically conductive distal spacer member 142 extends between the distal end 122 of the central electrode 120 and the distal end 132 of the electrode 130. Likewise, a proximal non-electrically conductive spacer 144 extends between the proximal end 114 of the electrode 110 and the proximal end 124 of the central electrode 120. Another non-electrically conductive proximal spacer member 146 extends between the proximal end 124 of the central electrode 120 and the proximal end 134 of the electrode 130. The spacer members 140, 142, 144, 146 may be fabricated from, for example, Teflon®.

Also in various embodiments, a spreader assembly 100 is attached to each of the proximal ends 114, 124, 134 of the electrodes 110, 120, 130, respectively. For example, the spreader assembly 150 may be fabricated from Teflon® and include a first spreader finger 152 that is attached to the proximal end 114 of the electrode 110 and a second spreader finger 154 that is attached to the proximal end 124 of the electrode 120. A third spreader finger 156 is attached to the proximal end 134 of the electrode 130. The first, second, and third spreader fingers 152, 154, 156 may be attached to the proximal ends of the electrodes 110, 120, 130, respectively by, for example, spring steel. The spreading mechanism 150 is fabricated to enable the electrodes 110, 120, 130 to be simultaneously inserted in through the catheter 50 and then when the spreader assembly 150 is advanced out through the distal end of the catheter 50, the spring steel spreader assembly 150 causes the electrodes 110, 120, 130 to spread apart from each other a distance defined by the lengths of each of the non-electrically conductive spacer members 140, 142, 144, 146. In various embodiments, the centrally-disposed electrode member 110 has a positive polarity, while the laterally-disposed electrodes 120, 130 each have a negative polarity.

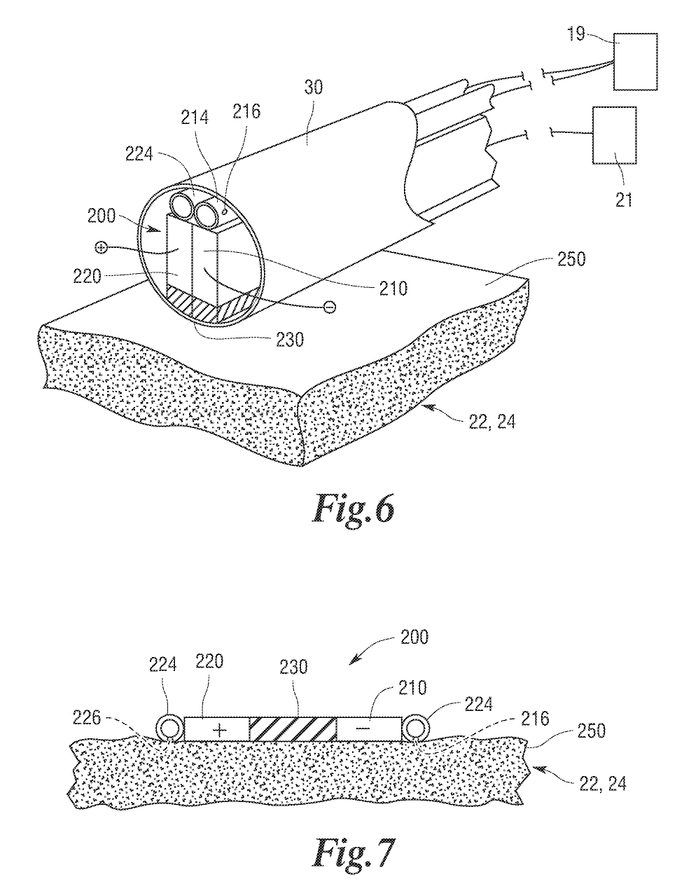

FIGS. 6 and 7 illustrate another probe assembly 200 that includes two electrodes 210, 220. Each electrode 210, 220, for example, may be 1.0 cm long×0.5 mm wide×01.mm thick and be fabricated from stainless steel. The electrodes 210, 220 may hinged together and attached to each other by a non-conductive hinge member 231. Hinge member 231 may be fabricated from a non-electrically conductive material. Such arrangement permits the probe assembly 200 to be deployed through the flexible catheter 30 in a first “folded” position (FIG. 6) wherein the electrodes 210, 220 are in a non-coplanar position. Once the probe assembly 200 has been deployed onto the target tissue 22, 24 it can be moved to a second “open” position wherein the electrodes 210, 220 are substantially co-planar. See FIG. 7.

The first and second electrodes 210, 200 are coupled to a source of electrical power 21 that may be located in the surgical suite. The first electrode 210 may have a positive polarity and the second electrode 220 may have a negative polarity. Attached along one lateral side of each electrode 210, 220 is a vacuum tube. More specifically for example, the first electrode 210 may have a first vacuum tube 214 attached to a first lateral side 212 that has a series of holes 216 therethrough. Similarly, the second electrode 220 may have a second vacuum tube 224 attached to a lateral side 222 that has a series of holes 226 therethrough. The vacuum tubes 214, 224, are attached to a source of vacuum 19 in the surgical suite.

In use, the probe assembly 200 is advanced through a flexible catheter 30 in a first folded position until the probe assembly 200 is adjacent the target tissue 22, 24. Thereafter, the probe assembly 200 is deployed to the open position wherein the electrodes 210, 220 can lie down on the epicardium tissue 250 as shown in FIG. 7. The vacuum applied through the holes 216, 226 in the vacuum tubes 214, 224, respectively serves to hold the electrodes 210, 220 in place on the epicardium 250 while the heart continues to beat. Pulses on the order of approximately 3000V, 1.0 μsec are applied to the epicardium 250. For example, a tissue damage having a depth of approximately 0.5 mm to 2.0 mm may be possible.

Those of ordinary skill in the art will recognize that the various embodiments of the devices and methods of the present invention represent alternatives to the prior approaches used to address atrial fibrillation. Such unique and novel devices and approaches of the various embodiments of the present invention may be effectively employed through a patient's natural orifice and thus avoid the complications and disadvantages often encountered when employing prior methods. While the various embodiments of the present invention have been herein described in connection with the treatment of atrial fibrillation, those of ordinary skill in the art will recognize that the various embodiments and methods of treatment and access of the present invention may also be successfully employed to treat other tissues and portions of the body. For example, the various embodiments and methods of the present invention could be employed to treat lymph nodes of the lungs and other structures.

While the embodiments have been described, it should be apparent, however, that various modifications, alterations and adaptations to the embodiments may occur to persons skilled in the art with the attainment of some or all of the advantages of the invention. For example, according to various embodiments, a single component may be replaced by multiple components, and multiple components may be replaced by a single component, to perform a given function or functions. This application is therefore intended to cover all such modifications, alterations and adaptations without departing from the scope and spirit of the disclosed invention as defined by the appended claims.

The devices disclosed herein can be designed to be disposed of after a single use, or they can be designed to be used multiple times. In either case, however, the device can be reconditioned for reuse after at least one use. Reconditioning can include a combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility.

Those of ordinary skill in the art will appreciate that the devices disclosed herein may be provided in a kit that may, for example, be directed to a particular surgical procedure. For example, a kit may include a probe assembly 50, 100, 200 in combination with flexible catheters 30, 40. The kit may further include an electrical generator, cryogenerator, etc.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An ablation probe assembly for treating cardiac tissue, comprising:
a first elongated electrode probe having a first polarity; and
a second elongated electrode probe having a second polarity that is opposite from said first polarity, said second elongated electrode probe coupled in spaced relation to said first elongated electrode probe by a first non-electrically conductive member to facilitate movement of said first and second elongated electrode probes relative to each other.

2. The ablation probe assembly of claim 1 further comprising a third elongated electrode probe coupled in spaced relation to said first elongated electrode probe by a second non-electrically conductive member to facilitate movement of said first and third elongated electrode probes relative to each other.

3. The ablation probe assembly of claim 2 further comprising a non-electrically conductive spreader assembly coupled to said first, second, and third elongated electrode probes for selectively moving said first, second, and third elongated electrode probes between a first position wherein said first, second, and third elongated electrode probes may be deployed through a flexible catheter and a second position wherein said first, second, and third elongated electrode probes are deployed in spaced relation to each other.

4. The ablation probe assembly of claim 1 further comprising:
a first vacuum tube coupled to said first elongated electrode probe; and
a second vacuum tube coupled to said second elongated electrode probe.

5. The ablation probe assembly of claim 1 further comprising an elongated flexible housing non-movably supporting said first and second elongated electrode probes therein in spaced-relation to each other, said elongated flexible housing being formed from non-electrically conductive material.

6. The ablation probe assembly of claim 5 further comprising at least one vacuum passage integrally formed in said elongated flexible housing.

7. The ablation probe assembly of claim 5 further comprising a third elongated electrode probe non-movably supported in said elongated flexible housing such that a first vacuum passage is integrally formed between said first and second elongated electrode probes and a second vacuum passage is integrally formed between said first and third elongated electrode probes.

8. A surgical kit for treating cardiac tissue, said surgical kit comprising:
an ablation probe assembly according to claim 1; and
a flexible catheter.

9. A method for treating cardiac tissue, said method comprising:
forming an incision through a patient's esophagus adjacent a posterior portion of the patient's heart;
inserting an ablation probe through the incision in the esophagus; and
activating the ablation probe to ablate a portion of heart tissue.

10. The method of claim 9 wherein said inserting comprises inserting the ablation probe through the patient's mouth and into the patient's esophagus.

11. The method of claim 9 wherein said ablating comprises applying electrical pulses to the cardiac tissue.

12. The method of claim 9 wherein said ablating comprises applying radio frequency pulses to the cardiac tissue.

13. The method of claim 9 wherein said ablating comprises:
inserting a cryoprobe through the incision in the esophagus;
contacting the cardiac tissue with the cryoprobe; and
activating the cryoprobe to irreversibly damage the cardiac tissue.

14. The method of claim 9 further comprising:
inserting a balloon catheter through the incision in the patient's esophagus prior to said inserting said ablation probe;
expanding the balloon catheter to establish space between the heart and the esophagus;
deflating the balloon catheter; and
removing the balloon catheter from the patient's esophagus.

15. The method of claim 9 wherein said ablating comprises:
contacting the cardiac tissue with the ablation probe; and
maintaining the ablation probe in constant contact with the cardiac tissue during said ablating.

16. The method of claim 15 wherein said maintaining comprises applying a vacuum to the cardiac tissue to draw the cardiac tissue into constant contact with the ablation probe.

17. The method of claim 14 further comprising:
inserting a flexible catheter through the incision after removing the balloon catheter from the patient's esophagus;
inserting the ablation probe through said flexible catheter;
contacting the cardiac tissue with the ablation probe; and
activating the ablation probe to ablate the cardiac tissue in contact therewith.

18. An ablation probe for ablating cardiac tissue, comprising
an elongated flexible housing sized to be inserted through a flexible catheter, said elongated flexible housing fabricated from non-electrically conductive material and having first and second separate vacuum passages formed therein;
a first positive electrode disposed within said elongated flexible housing;
a second negative electrode disposed within said elongated flexible housing such that said first said vacuum passage separates said first and second electrodes; and
a third negative electrode disposed within said elongated flexible housing such that said second vacuum passage separates said first and third electrodes.

19. An ablation probe for ablating cardiac tissue, comprising
a first elongated electrode having a first polarity;
a second elongated electrode having a second polarity that is opposite from said first polarity; and
a non-conductive hinge member coupled to said first and second elongated electrodes such that said first and second elongated electrodes may be moved between a first position wherein said first and second electrodes are adjacent to each other in a non-coplanar relationship and a second position wherein said first and second electrodes are substantially coplanar with each other.

20. The ablation probe of claim 19 further comprising:
a first vacuum tube coupled to said first elongated electrode; and
a second vacuum tube coupled to said second elongated electrode.
